(19) United States
(12) Patent Application Publication
(10) Pub. No.: US 2026/0072035 A1
(43) Pub. Date: Mar. 12, 2026

(54) ENHANCED SPECIFICITY MASS TAG DNA ADDUCTOMICS

(71) Applicant: Northeastern University, Boston, MA (US)

(72) Inventor: Roger W. Giese, Hanover, MA (US)

(21) Appl. No.: 18/867,499

(22) PCT Filed: May 31, 2023
(86) PCT No.: PCT/US2023/023962

§ 371 Date: Nov. 20, 2024

Related U.S. Application Data
(60) Provisional application No. 63/347,790, filed on Jun. 1, 2022.

(51) Int. Cl. G01N 33/58

(52) U.S. Cl. CPC G01N 33/58; G01N 2560/00

(57) ABSTRACT

Certain combinations of amine-targeting mass tags with techniques selected from strong anion exchange, mild depurination, and affinity chromatography give advanced dna adductomics.

### RELATED APPLICATIONS

This application claims the benefit of priority to U.S. Provisional Patent Application Ser. No. 63/347,790, filed Jun. 1, 2022.

### BACKGROUND

Cancer Prevention Test. While there is a heart attack prevention test (cholesterol), there is no corresponding cancer prevention test. Over 50 years ago cancer epidemiologists proposed that a test for DNA adductomics (covalently-damaged nucleotides in DNA) should be useful for cancer prevention: the person learns their elevated adducts, and then changes their environment (defined in the broadest sense) to lower these adducts and thereby lower cancer risk. In spite of thousands of articles reporting many methods since then on DNA adducts, an effective DNA adductomics test for cancer prevention does not exist.

### SUMMARY OF THE INVENTION

This invention changes that, by introducing a new kind of test for DNA damage, termed Enhanced Specificity Mass Tag DNA Adductomics (“ESMD”) that precisely and broadly defines genotoxic exposure in a practical way. This includes its use for therapeutic drug monitoring of chemotherapeutics to optimize the therapy in terms of maximizing efficacy, while minimizing side effects and second can cancer. ESMD thereby helps to prevent both primary and secondary cancer.

### BRIEF DESCRIPTION OF THE FIGURES

FIG. 1. Scheme for detection by ESMD of damaged nucleobases or nucleosides in urine (or derived from DNA or RNA in urine), along with extension to cancer prevention.

FIG. 2. Three preferred sides of guanine or guanines [the concept also applies to adenine(s)] for recognition by a complementary affinity reagent: I, II, or III. Other sides of guanine or guanines can be targeted, for example, the side comprising the O6 and N7 sites, or the side comprising the O⁶, N7 and C8 sites. Further potential affinity targeting: part of a modification on a guanine; or part of such modification along with part of the guanine.

FIG. 3. Line drawing of an affinity complex of guanine and a bidentate affinity ligand (derived from a diacylphenyl type of affinity ligand template) recognizing the II side of guanine(s), where the affinity ligand is leashed to a solid phase particle (which particle is indicated by the wavy line). A doubly alkene-modified form of this affinity ligand (not shown) can be built into a molecular imprinted polymer, as by using the overall strategy of Poma et al. (Poma et al., [2014] Nucleoside-Tailored Molecularly Imprinted Polymeric Nanoparticles (MIP NPs), Macromolecules, 47, 6322-6330) FIG. 4. Space-filling model of the complex shown in FIG. 3 to achieve a solid phase affinity reagent.

FIG. 5. Formation of reactive and liganded silica particles. This figure is replicated from Poma et al. (2014) Nucleoside-Tailored Molecularly Imprinted Polymeric Nanoparticles (MIP NPs), Macromolecules, 47, 6322-6330).

FIG. 6. Proposed structure of a complex between β-cyclodextrin and guanosine based on NMR analysis. This figure is replicated from Prabu, et al. (2020) Studies on the supramolecular complex of a guanosine with β-cyclodextrin and evaluation of its anti-proliferative activity, Carbohydrate Research, 497, 1081.38

FIG. 7. Labeling of N7-guanines with CAX-CHO for detection by mass spectrometry.

FIG. 8. Labeling of 1,N⁶-ethenoadenines with CAX-B for detection by mass spectrometry.

### DETAILED DESCRIPTION

Urine. Urine is a preferred sample for this invention. It is noninvasive even when scaled up, and can contain DNA adducts from throughout the body. While DNA adducts can be at a low level in urine, labeling them especially in a nucleobase form with a N-targeting quaternary amine mass tag reagent, preferably a CAX (cationic xylyl) mass tag, boosts their signal strength in a mass spectrometer. Other types of mass tags can be used as well such as those possessing a tertiary amine, phosphonium or pyridinium group providing a positive charge.

Surrogate Testing. Each genotoxic chemical tends to give multiple DNA adducts not only by reacting with different monomers of the DNA (the common nucleotide, nucleoside or nucleobase components of a nucleic acid), but also by reacting with multiple sites on some monomers. To discover or assess a given genotoxic exposure, only one of these adducts from a given genotoxic chemical, or adduct type (as a surrogate) needs to be measured.

Nevertheless, the ESMD test can measure multiple adduct classes in a given procedure, or in multiple procedures, to broaden the genotoxicity information acquired. Selecting a surrogate class that reflects many exposures, with a different adduct characteristic of each exposure, is advantageous. One class that does this consists of N7-guanine adducts (N7-guanines). This is because many DNA-damaging chemicals especially attack the highly reactive N7 position of guanine. EDMD readily measures this class of adducts, and also other adduct classes.

Purine Focus. Purine nucleobase adducts are preferred for detection. Such adducts are readily formed by genotoxic chemicals; easy to harvest from DNA (by mild neutral thermal hydrolysis or mild acid depurination) as nucleobases, and can be efficiently labeled with a mass tag for sensitive detection by mass spectrometry. Especially preferred are N7-guanine nucleobase adducts (N7-guanines) that are formed predominantly by many genotoxic chemicals, and are readily released from DNA by mild neutral hydrolysis since they have a positive charge. Other preferred purine nucleobase adducts for the same reason are N3-guanines, N7-adenines, and N3-adenines. Certain pyrimidine adducts that are hydrolytically unstable on DNA are preferred as well for measurement by ESMD, including O²-cytosines and O²-thymines. Neutral purines including adducts thereof can be isolated from DNA by mild acid hydrolysis. Measurement of the canonical purines in this procedure nicely enables the amount of DNA to be determined. The ESMD method also can be applied to detection of DNA adducts in a nucleoside form, obtained from DNA by enzymatic digestion.

Practical Cancer Prevention Test. In order to stimulate significant interest by cancer epidemiologists and enter routine clinical diagnostics, a DNA damage test for cancer prevention must be practical. The methodology presented here for detection of nucleomer adducts is practical, in part by employing one or more affinity reagents to give the resolution required in a simple way, as a replacement for high performance liquid chromatography (HPLC). HPLC is a common step at the preparation or detection stage in many current, valuable methods for DNA adductomics, but this technique tends to be complicated, time-consuming, delicate, and expensive, making such methods impractical. Instead short-column chromatography-MS, infusion-MS, or MALDI-MS techniques, which are practical and fast, are used at the detection stage of ESMD, helping to make this technique practical.

Impact of a Cancer Prevention Test. The environment, defined in the broadest sense, is a major cause of cancer. The impact of food genotoxicity information would be greater if it were personalized, for example as follows: (1) a person learns from a ESMD test that they have elevated damage to their DNA (elevated DNA adducts, “dirty DNA”) from their diet; (2) they change their diet accordingly to include more healthy options; (3) the amount of damaged adduct decreases; (4) this personal feedback of achieving cleaner DNA motivates them to continue with their new diet; and (5) their cancer risk goes down. An analogy is a person with diabetes who is motivated to take their medicine and practice a low-carb diet when this lowers their HbAlc level, a sign that their diabetes is under better control. Personalized cancer prevention information can be more powerful than general cancer prevention information.

Aptamers. Aptamers can be useful as affinity reagents in ESMD to help purify DNA adducts for detection (see below). Aptamers are known which bind DNA adducts (McKeague, M. [2017] Aptamers for DNA Damage and Repair. Int. J. Molecular Sciences, 18, 2212-2228.) An aptamer has been prepared that binds guanine and xanthine (Kiga, D., Futamura, Y., Sakamoto, K., Yokoyama, S. [1998] An RNA aptamer to the xanthine/guanine base with a distinctive mode of purine recognition. Nucleic Acids Res., 26, 1755-1760). Aptamers for use in this invention can be developed and acquired commercially, as from Aptagen.

Chromatographic Retentive Power of Multi-H-Binding. The high retentive power of a multi-H-bonding ligand interactions in chromatography has been demonstrated using an N-[7-(2,4-dimethyl-1,8-naphthyridyl)] ligand, which gave high retention for a guanosine derivative (Feibush, B., Saha, M., Onan, K., Karger, B., Giese, R. [1987] HPLC Separation of DNA Adducts Based on Hydrogen Bonding. J. Am. Chem. Soc. 109, 7531-7533). This is an example of partner affinity chromatography, where the binding partner for the substance of interest is similar in size to this substance.

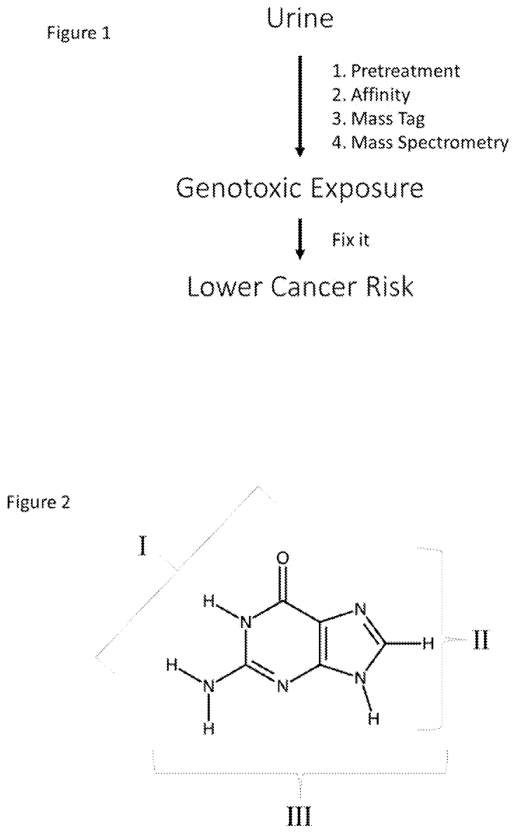

DNA Nanopore Voltage-Driven Filtration. DNA species (DNA nucleotides or DNA oligonucleotides) are driven through a narrow channel provided by a channel protein, where the protein sits in a membrane; a solution of a DNA species is above the membrane; an appropriate voltage is applied across the membrane; and the DNA species goes through the channel. The channel can be very narrow, e.g., 90% of its volume can be occupied by a segment of the DNA species as it goes through. DNA nanopore voltage-driven filtration provides DNA nanopore sequencing when the current is monitored for a pore. DNA nanopore voltage-driven filtration is useful in ESMD as part of sample pretreatment (see FIG. 1). Preferred is to use it on 100-mers, where nonadducted 100-mers pass through the channel quickly, while 100-mers having a bulky adduct get stuck. Reversing the voltage then recovers DNA enriched in bulky adducts.

DNA Repair Enzymes. DNA repair enzymes are useful in this invention, especially DNA glycosylases, as part of sample pretreatment, to release DNA adducts as modified nucleobases from DNA. DNA glycosylases have been reviewed (Lindahl, T. [1979] DNA glycosylases, Endonuclease for Apurinc/Apyrmidinic Sites and Base Excision-Repair. Progress in Nucleic Acid Research and Molecular Biology 22, 135-192).

Quaternary Amines. For the mass tag of FIG. 1, a positively charged reagent is preferred, especially a quaternary amine, and most preferred is a CAX (cationic xylyl) mass tag, of two types. The first type is CAX-CHO, to label an exocyclic primary amino group on a damaged nucleomer in the presence of a hydride reagent, especially sodium cyanoborohydride or pyridine borane. The second type is CAX-B (cationic xylyl bromide), to label a secondary amine site on a modified nucleomer.

Technical Considerations. While urine can contain damaged nucleomers from the diet, a dietary source for these can be defined or controlled to rule out or define diet as a source or potential source of a modified nucleomer in the sample being tested. For example, this can be done by changing the diet and retesting.

Damaged nucleomers detected in urine as damaged nucleobases are preferred for detection in this invention. They may come from RNA or DNA. The simultaneous measurement of modified nucleomers from both DNA and RNA is consistent with the goal of defining genotoxic exposure.

Recovery of Purines. The initial urine can be treated with ribonucleases (e.g. RNAse A and RNAse T1) to remove RNA, and the remaining DNA can be purified from the urine, as by ultrafiltration or precipitation, prior to starting an acidic depurination reaction. This procedure, in addition to the above technique (in Technical Considerations), can eliminate diet and RNA as a source of modified nucleomers detected in the method. Positively charged adducts in the DNA, such as N7-guanines or N3-adenines, can be recovered from DNA by mild neutral thermal hydrolysis followed by CAX labeling/MS detection. Neutral purine nucleomers next can be recovered from the DNA by mild acid hydrolysis followed by similar detection, and this nicely gives a way to quantify the amount of DNA in the sample. ESMD can be applied to 24 hour urine samples.

Biosamples to be Tested. While urine is the preferred sample for this detection method, any kind of biosample containing DNA can be tested. Kits are available to isolate DNA from biosamples in general.

CAX Mass Tags. The labeling reaction with a CAX mass tag is important in two general ways. First, this increases the response of adducts up to about 1000-fold in the mass spectrometer, enabling a biosample such as urine, which has a low concentration of DNA, to be tested. Second, this helps to achieve comprehensive detection of DNA adducts, since, without this labeling, adducts with a lower inherent sensitivity in the mass spectrometer are not detected.

DNA Adducts and DNA Adductomics. Hundreds of scientists have contributed thousands of publications on the measurement of DNA adducts (damaged nucleotides in DNA) both as single adducts, and as groups of adducts (DNA adductomics) over the past 50 years. DNA adducts can “turn into” mutations, and mutations are at the heart of all cancer. The primary goals of DNA adduct analyses are to better understand cancer, and to help to prevent it by learning what causes DNA it. ESMD is important for other conditions such as diabetes and aging.

Many DNA Adductomics Methods. The many methods for measuring DNA adducts vary in their convenience, specificity, scope, and cost. Overall, DNA adductomics today largely is a failure. There is nowhere in the world where one can send a biosample for good DNA adduct testing: low cost, specific, rugged, quantitative, practical and broad scope.

ESMD Enhances Prior Adductomics. The prior adductomics techniques of Jettison Mass Spectrometry (Wang, P., Shah, G. L., Landau, H., Coulter, M. E., Walsh, C. A., Roider, E., Kramer, C. S., Beuning, P. J., Giese, R. W. [2020] Jettison-MS of Nucleic Acid Species, J. Am. Soc. Mass Spectrom. 31, 1641-1646) and Prelabeling (Wang, P., Roider, E., Coulter, M. E., Walsh, C. A., Kramer, C. S., Beuning, P. J., Giese, R. W. [2021] DNA Adductomics by mass tag prelabeling, Rapid Commun. Mass Spectrom. 35: e9095) are brought to a new level of performance by ESMD. In both cases, the aldehyde mass tag labeling reaction can be applied directly to the DNA as a first step, and then the usual steps follow. One can also subject a DNA sample to enzymatic digestion that yield nucleosides, and apply the aldehyde mass tag at that point, or after partner affinity purification is applied to the DNA digest.

### Definitions of Terms

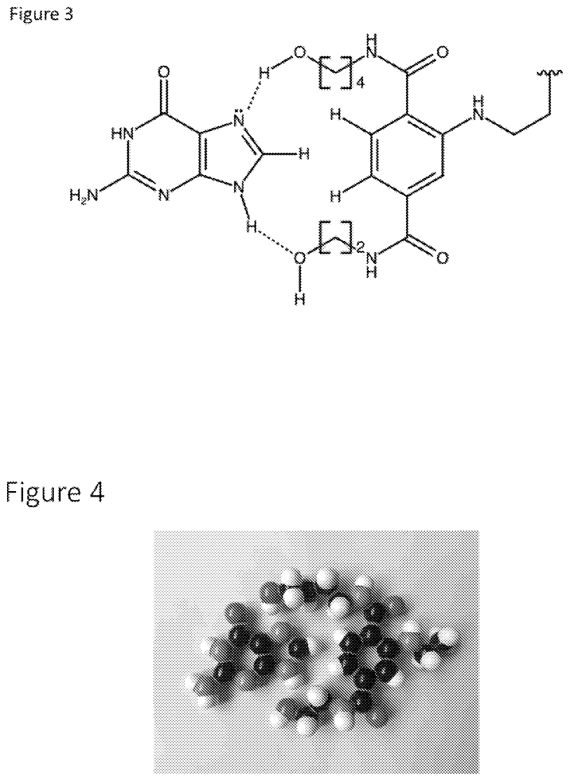

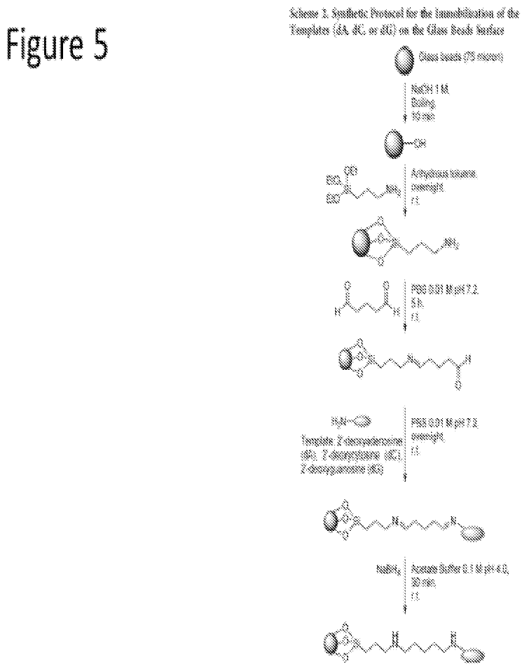

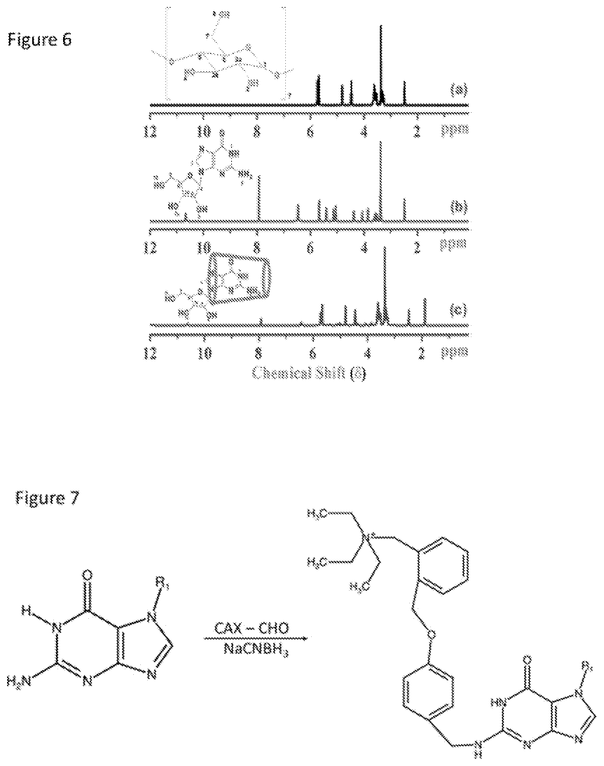

Canonical Nucleomer: adenine, adenosine, 2′-deoxyadenosine, guanine, guanosine, 2′-deoxyguanosine, cytosine, cytidine, 2′-deoxycytidine, 5-methycytosine, 5-methylcytidine, 5-methyl-2′-deoxycytidine, thymine, thymidine, uracil, uridine or a naturally-modified form of one of these contributing to the natural structure or function of a nucleic acid molecule.
Noncanonical Nucleomer: a canonical nucleomer that is chemically modified in a non-natural way (causing loss of natural function or structure), as by alkylation, acylation, amination, halogenation, hydrolysis, radical attack, electromagnetic radiation, or oxidation, including products arising from a combination of these events, and also products from molecular rearrangements due to such events. Other terms here for a noncanonical nucleomer are “adduct”, “nucleomer adduct”, “damaged nucleomer”, “modified nucleomer”, “DNA adduct”, or “RNA adduct”.
Targeted Nucleomer: a nucleomer to be detected in a given procedure.
Nontargeted Nucleomer: a nucleomer not to be detected in a given procedure.
Nucleomer: a canonical or noncanonical nucleomer.
Nucleomer Component: a canonical or noncanonical nucleomer which is part of DNA, RNA, or of a nucleotide of the DNA or RNA type.
Guanines: noncanonical guanines such as N7-guanines.
Adenines: noncanonical adenines such as N3-adenines.
Canonical Nucleobase: A nucleomer in the form of a nonmodified nucleobase such as adenine, guanine, cytosine, 5-methylcytosine, thymine, or uracil.
DNA: genomic DNA, fragmented DNA, or DNA oligomer
Noncanonical Nucleobase: A nucleobase that has been chemically modified in a non-natural way as above (see Noncanonical Nucleomer).
Noncanonical DNA: DNA that has been chemically modified in a nonnatural way; also referred to as “adducted DNA”.
Noncanonical RNA: RNA that has been chemically modified in non-natural way (a way that is not natural in nature).
Nucleobase: a nucleobase that is either canonical or noncanonical.
Nucleoside: a canonical or noncanonical nucleoside, in a deoxy form (characteristic of DNA) or in a nondeoxy form (characteristic of RNA).
Nucleobase: a purine or pyrimidine that is either canonical (e.g. adenine, guanine, thymine, cytosine, uracil, 5-methylcytosine, or a naturally-modified form of one of these) or noncanonical.
Nucleoside: a sugar-purine or sugar-pyrimidine compound, where the sugar is either ribose or deoxyribose, and the compound is either canonical or noncanonical.
Purines: guanines and adenines.
Biosample: a biological or physiological fluid, cells, aerosol, or tissue including urine, blood, saliva, cerebrospinal fluid, white blood cells, plasma, skin, hair, placenta, semen, stool, liver, sweat, tears, breath, stool, lung, or liver. Urine is preferred.
Biosample Extract: what is recovered in, on, or through a liquid or solid phase second material after a biosample as a first phase material is treated with this second phase material, where the treatment process comprises a purification step such as solid phase extraction, liquid phase extraction, partitioning, centrifugation, precipitation, redissolving, or liquid chromatography.
Affinity Reagent: a dissolved or solid phase material providing two or more attractive interaction sites, as via an affinity ligand, for its target molecule (a noncanonical nucleomer to be detected, or an interfering nucleomer to be removed) thereby enabling convenient purification of a noncanonical nucleomer of interest prior to its detection. Examples of types of affinity ligands or reagents useful in this invention are bidentate diacyphenyl-templated (one option is shown in FIGS. 3, 4); (part of FIG. 5); β-cyclodextrin (FIG. 6, e.g. employ DEXSORB from CycloPure); a molecular imprinted polymer (MIP, e.g. prepared using imidazole or guanine as a ligand, which ligand may be leashed to a solid support for preparation of the MIP); nucleic acid aptamer; peptide aptamer (peptimer); nanobody, antibody, aptabody, vacancy G-quadruplex scaffold, imidazolium based cyclophane, or riboswitch. Affinity reagents for guanine have been reviewed (Li, Y., Liu, J., [2020] Sensing guanine and its derivatives: From molecular recognition to applications. Sensors and Actuators Reports 2, 100020). Examples of noncanonical nucleomer affinity ligands are 8-oxoguanine (recognizes adenine) and hypoxanthine (recognizes cytosine). A polymeric form of an affinity ligand can be used, e.g. polycytidylic acid bound to a strong anion exchange resin. Affinity reagents tend to be more retentive for their target molecule when used at a lower temperature, e.g. 5° C. The temperature can even be lowered to below the freezing point of water by adding an organic solvent, such as acetonitrile or methanol.
Affinity Ligand Template: A molecule or molecular group which is extended by synthesis to yield an affinity ligand. For the affinity ligand shown in FIGS. 3, 4, the template is 2-nitrophthaloyl chloride, a diacylphenyl type of affinity ligand template. Other examples of affinity ligand templates for use in this invention are 2-nitro-para-phenylenediamine, and 2-fluoro-1,3-diaminobenzene derived from 2-fluoro-1,3-dinitrobenzene.
Partner Affinity: An affinity step in which the reagent binding partner for a nucleomer of interest is of similar size as the nucleomer, such as less than about two-fold larger.
Biomarker: A substance derived from an animal, plant, or human whose measurement helps to answer a question about health or disease.
Semipolar Solid Phase Extraction: A semipolar chromatographic bed (e.g. bonded silica, OASIS) used to retain nonpolar analytes while polar interferences are washed out, or to retain nonpolar interferences while polar analytes are washed out. Semipolar solid phase extraction can be useful in this invention as an affinity reagent, based on its providing simultaneous nonpolar and polar attractive contacts with a target molecule of interest. Even methylated purines, which are only slightly nonpolar, can be retained by semipolar extraction (Still, W. G., Xu, H.-X., Adkins, J. A., Wishnok, J. S., Tannenbaum, S. R. [1989] Analysis of Methylated and Oxidized Purine in Urine by Capillary Gas Chromatography-Mass Spectrometry. Chem Res. Toxicol. 2, 94-99), and this can be enhanced at a low temperature such as 5° C.
Mixed-mode Solid Phase Extraction: A mixed-mode solid phase extraction chromatographic bed used to retain nonpolar analytes while polar interferences are washed out, or to retain nonpolar interferences while polar analytes are washed out. Semipolar solid phase extraction can be useful as an affinity reagent, based on its providing simultaneous, divergent, attractive contacts, such as nonpolar and ionic, with a target molecule of interest. An example is provided by Hu et al. (Hu, K., Zhao, G., Liu, J., Xie, F., Zhang, S., Liu, H., Liu, M., [2018] Simultaneous quantification of three alkylated purine adducts in human urine using sulfonic acid poly(glycidyl methacrylate-divinlbenzene)-based microsphere as sorbent combined with LC-MS/MS. J. Chromatogr. B, 1082, 15-24). This technique can be enhanced by operating it at a low temperature such as 5° C.
Mass Spectrometry: A form of mass spectrometry for organic substances, such as liquid chromatography/electrospray/Orbitrap mass spectrometry; liquid chromatography/electrospray/triple quadrupole mass spectrometry; liquid chromatography/electrospray/ion trap mass spectrometry; or MALDI-TOF mass spectrometry (including MALDI-TOF/TOF and TIMS-TOF-MS).
Exemplary Nucleoside Procedure: DNA or RNA that has been enzymatically digested to give nucleosides, subjecting the nucleosides to chromatography such as affinity chromatography, labeling a targeted nucleoside possessing a primary amino group with a positively charged reagent possessing a reactive aldehyde group in the presence of a hydride reagent, and detecting the labeled nucleoside by mass spectrometry.

### Methodology

Below are presented nine illustrative measurement procedures (designated as Methods A-L), some of which take advantage of one or more affinity reagents, to detect a noncanonical nucleomer. In some cases, a particular type of damage is considered as an example. Conventional extraction steps such as chromatography, partitioning, centrifugation, or filtration can be part of each measurement procedure, especially in the pretreatment stage (see FIG. 1) to help purify a given type of noncanonical nucleomer analyte from background substances in the biosample. Each procedure may detect one or more nucleomers because final detection is by mass spectrometry.

Method A: Detection of N7-guanine Adducts (N7-guanines) and Application to Cancer Prevention
Step 1. Urine is collected on one or more occasions into standard, screw-top, urine collection cup(s), and the urine-containing cup(s) is stored in a freezer, or preserved in another way for storage at room temperature, such as by adding rubbing alcohol. Urine can also be collected in a jar or bottle.
Step 2. The urine is warmed and kept as such to enable spontaneous depurination to take place of the N7-guanines, such as conditions of three days at 37° C., pH 7.0 (mild neutral thermal hydrolysis) yielding N7-guanines.
Step 3. Guanine(s) unsubstituted on the I side (see FIG. 2) are affinity extracted by base-pairing using a cytosine-substituted chromatographic material, such as the cytosine affinity material option in FIG. 5, which recognizes the I side of guanines (see FIG. 2). Not much of guanine may be encountered since it resists formation in neutral thermal hydrolysis. A β-cyclodextrin ligand can be employed for affinity extraction of guanine(s) (FIG. 6 shows a guanosine/β-cyclodextrin complex.)
Step 4. The sample optionally is treated with adenosine deaminase to render potentially-interfering adenine or adenines unreactive with an aldehyde-functionalized mass tag in a subsequent step.
Step 5. Nontargeted guanine(s) optionally are removed by affinity chromatography using a ligand-substituted chromatographic material where the ligand recognizes side II (see FIG. 2) of guanines.
Step 6. Label the recovered N7-guanines with a CAX-CHO mass tag as shown in FIG. 7. CAX-CHO-2 also can be used.
Step 7. Remove residual CAX-CHO mass tag by reaction with a chromatographic material bearing a primary alkyl amine or aryl amine or hydrazide group, such as the amine chromatographic material shown in FIG. 5, or ethylenediamine-polyacrylamide, in the presence of a hydride reagent such as sodium cyanoborohydride or pyridine borane.
Step 8. Detect the CAX-labeled N7-guanines by Mass Spectrometry.
Step 9. Inform the urine-donor about any elevated adducts so they potentially can reduce their risk for cancer by lowering their exposure to the chemical(s) or conditions that cause these adducts. (If this instead is a follow-up [second] test, and they have already modified their exposure resulting in lowered adducts, this feedback can encourage them to continue their improved behavior and thereby sustain their lower cancer risk.)

It is recognized that the order of some of the steps can be changed; and that analogous reagents and conditions can be employed.

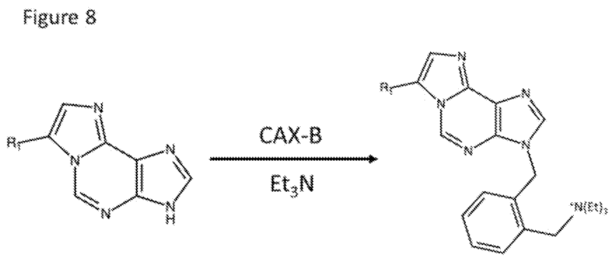

Method B: Detection of O⁶-guanine Adducts (O⁶-guanines) and application
Step 1. Same as Step 1 in Method A.
Step 2. The urine is dethawed, treated with acid to lower its pH to 5.0, and heated at 45° C. for 8 hours to give depurination of noncanonical DNA, DNA-oligos, deoxynucleotides, and corresponding RNA species, yielding O⁶-guanines.
Step 3. Guanines unmodified on their I side (see FIG. 2) are affinity removed by base-pairing using a cytosine-substituted chromatographic material, where the cytosine affinity ligand is leashed via its exocyclic amino group, one of the options of FIG. 5.
Step 4. O⁶-guanines and adenines lacking substitution on the II side (see FIG. 2 where this is illustrated for guanine) are affinity extracted by an affinity reagent directed at the II region, such as one employing the affinity ligand of FIGS. 3 and 4.
Step 5. The sample optionally is treated with adenosine deaminase to render potentially interfering adenines bearing a primary amine group unreactive towards an aldehyde-bearing mass tag reagent in a subsequent step.
Step 6. Label the O⁶-guanines with a CAX-CHO mass tag as shown in FIG. 7 for N7-guanines.
Step 7. Remove residual CAX-CHO mass tag by reaction with a chromatographic material bearing a primary amine group, such as the primary amine chromatographic material shown in FIG. 5 (Poma et al.)
Step 8. Detect CAX-labeled N7-guanines by mass spectrometry.
Step 9. Same as in Step 9 of Method A.
   Method C: Detection of N2-guanines and Application
Step 1. Same as Step 1 in Method A.
Step 2. The urine is dethawed, treated with acid to lower its pH to 5.0, and heated at 45° C. for 8 hours to give depurination of noncanonical DNA, DNA-oligos, deoxynucleotides, and corresponding RNA species, yielding purines including N²-guanines.
Step 3. Guanines unsubstituted in the I region (see FIG. 2) are affinity removed by base-pairing using a cytosine-substituted chromatographic material, where the cytosine ligand is leashed via its exocyclic amino group, one of the options of FIG. 5.
Step 4. Label the N²-guanines with a CAX-B mass tag in the presence of triethylamine as illustrated in FIG. 8 for 1,N⁶-adenines, using the conditions described by Wang et al., (Wang, P., Zhang, Q. Yao, Y., Giese, R. W. (2015) Cationic Xylene Tag for Increasing Sensitivity in Mass Spectrometry, J. Am. Soc. Mass Spectrom. 26, 1713-1721).
Step 5. Remove residual CAX-B by reaction with a chromatographic material bearing a nucleophilic group such as guanine (see FIG. 5), phenol, sulfhydryl, or imidazole overnight in the presence of triethylamine.
Step 6. Detect the CAX-labeled N²-guanines (N²-guanine adducts) by mass spectrometry.
Step 7. Same as Step 9 in Method A.
   Method D: Detection of C8-guanines and Application
Step 1. Same as Step 1 in Method A.
Step 2. The urine is dethawed, treated with acid to lower its pH to 5.0, and heated at 45° C. for 8 hours to give depurination of noncanonical DNA, DNA-oligos, deoxynucleotides, and corresponding RNA species, yielding purines including C8-guanines.
Step 3. Guanines unsubstituted in the I region are extracted using the cytosine-substituted chromatographic material of FIG. 5.
Step 4. The sample optionally is treated with adenosine deaminase to render potentially-interfering adenines bearing a primary amine group unreactive towards an aldehyde-bearing mass tag in a subsequent step.
Step 5. Label the C8-guanines with a CAX-CHO mass tag as shown in FIG. 7 for N7-guanines.
Step 6. Remove residual CAX-CHO mass tag by reaction with a chromatographic material bearing primary amine group, such as the amine chromatographic material shown in FIG. 5, in the presence of a hydride reagent.
Step 7. Detect the CAX-labeled C8-guanines by mass spectrometry.
Step 8. Same as in Step 9 of Method A.
   Method E: Detection of N3-adenines and Application
Step 1. Same as Step 1 of Method A.
Step 2. The urine is warmed and kept as such to enable spontaneous depurination to take place of the N3-adenines, such as conditions of three days at 37° C., pH 7.0 (mild neutral thermal hydrolysis) yielding N3-adenines.
Step 3. Label the N3-adenines with CAX-CHO. (Labeling of canonical guanine and adenine can be tolerated when little of these species is present.)
Step 4. Remove residual CAX-CHO by reaction with a chromatographic material bearing a primary amine group, such as the primary amine chromatographic material shown in FIG. 5, in the presence of a hydride reagent.
Step 5. Detect the CAX-labeled N3-adenines by mass spectrometry.
Step 6. Same as Step 9 in Method A.

Note: CAX-B also can be used to label N3-adenines for detection, followed by removal of residual CAX-B as in Method C.

Method F: Detection of 1,N⁶-adenines and Application
Step 1. Same As Step 1 in Method A.
Step 2. The urine is dethawed, treated with acid to lower its pH to 5.0, and heated at 45° C. for 8 hours to give depurination of DNA, DNA-oligos, deoxynucleotides, and corresponding RNA species, yielding purines including 1,N⁶-adenines.
Step 3. Guanines unsubstituted in the I region (see FIG. 2) are extracted using the cytosine-substituted chromatographic material of FIG. 5.
Step 4. Adenine is deaminated with adenosine deaminase.
Step 5. Label the 1,N⁶-ethenoadenines with CAX-B in the presence of triethylamine as illustrated in FIG. 8.
Step 6. Remove residual CAX-B by reaction with a chromatographic material bearing a nucleophilic group such as imidazole, phenol, sulfhydryl, or guanine (see FIG. 5 for guanine), overnight in the presence of triethylamine
Step 7. Detect the CAX-labeled 1,N⁶-ethenoadenines by mass spectrometry.
Step 8. Same as Step 9 in method A.
   Method G. Detection of N7-guanines From Noncanonical DNA in Urine and Application
Step 1. Treat the urine with ribonucleases to convert RNA species to nucleotides.
Step 2. Treat the urine with a strong anion exchanger such as Nuvia Q Anion Exchange Resin from Bio-Rad, to bind noncanonical DNA.
Step 3. With a low salt buffer, wash nucleomers, nucleotides, and background substances off the Resin, leaving noncanonical DNA still bound.
Step 4. Subject the resin to mild neutral thermal hydrolysis, or mild acid depurination, to release nucleomers to be detected as in the above corresponding methods, e.g. as in Method A (steps 3-9) when neutral thermal hydrolysis is done, or as in Method B (steps 3-9) when acid depurination is done.
Step 5. Same as Step 9 in Method A.

### Method H. Detection of DNA-protein Crosslinks in DNA in Urine and Application

Steps 1-3. Same as Method G.
Step 4. Using conditions reported by Barker et al, (Barker, S., Murray, D., Zhene, J., Li, L., Weinfeld, M/[2005] A method for the isolation of covalent DNA-protein crosslinks suitable for proteomics analysis. Anal. Biochem. 344, 204-215), subject the resin to aqueous chaotropes in the presence of Proteinase K and ribonucleases, followed by washing, mild acid depurination of the remaining noncanonical DNA still on the resin, and then follow steps 3-9 of Method B.
   Method I. Detection of 1,N⁶-adenines in Urine DNA and Application
Step 1. Urine is treated with ribonucleases and Proteinase K.
Step 2. DNA is isolated from the urine as described by Yun (Yun, B. H., Belliamri, M., Rosenquist, T. A., Turesky, R. J. [2018] A Method for Biomonitoring of DNA Adducts in Exfoliated Urinary Cell by Mass Spectrometry. Anal. Chem. 90, 9943-9950), or by ultrafiltration.
Step 3. The urine is treated with acid to lower its pH to 5.0, and heated at 45° C. for 8 hours to give depurination.
Step 4. 1,N⁶-adenines are recovered in the filtrate when the urine is subjected to ultrafiltration to remove residual noncanonical DNA.
Step 5. Guanines unsubstituted in the I region (see FIG. 2) are extracted using the cytosine-substituted chromatographic material shown in FIG. 5.
Step 6. Adenines are deaminated with adenosine deaminase.
Step 7. Label the 1,N⁶-ethenoadenines with CAX-B in the presence of triethylamine as illustrated in FIG. 8.
Step 8. Remove residual CAX-B by reaction with a chromatographic material bearing a covalently leashed nucleophilic group such as guanine (see FIG. 5), imidazole, phenol, or sulfhydryl overnight in the presence of triethylamine
Step 9. Detect the CAX-labeled 1,N⁶-ethenoadenines by mass spectrometry.
Step 10. Same as Step 9 in method A.
   Method J. Alternative Processing of Urine for Detection of N7-guanines.

Urine, as is or after treatment with a cell lysis reagent such as rubbing alcohol, is poured through a strong anion exchange chromatography column or filter, followed with rinsing with water and then rubbing alcohol. The column or filter is then warmed in buffer to release N7-guanines for additional steps leading to detection as described in Method A (steps 3-8).

### Method K. DNA Sample Testing

The DNA sample in saline is incubated with CAX-CHO in the presence of a hydride reagent. The DNA is precipitated with isopropyl alcohol, washed with aqueous isopropyl alcohol, and subjected to Jettison MS as described (Wang, P., Shah, G. L., Landau, H., Coulter, M. E., Walsh, C. A., Roider, E., Kramer, C. S., Beuning, P. J., Giese, R. W. [2020] Jettison-MS of Nucleic Acid Species, J. Am. Soc. Mass Spectrom. 31, 1641-1646).

### Method L. Mass Spectrometry Using CAX-CHO

CAX-CHO in the presence of a hydride reagent is used in place of CAX-B in the procedure described (Wang, P., Roider, E., Coulter, M. E., Walsh, C. A., Kramer, C. S., Beuning, P. J., Giese, R. W. [2021] DNA Adductomics by mass tag prelabeling, Rapid Commun. Mass Spectrom. 35: e9095), leading to mass spectrometry of CAX-CHO labeled nucleotides. The CAX-CHO-labeled nucleotides can also be digested to corresponding CAX-CHO labeled nucleosides prior to detection by mass spectrometry.

Synthesis of CAX-CHO-2. 3-Aminobenzaldehyde ethylene acetal (AlfaEasar) is reacted with CAX-B (Wang, P., Zhang, Q. Yao, Y., Giese, R. W. (2015) Cationic Xylene Tag for Increasing Sensitivity in Mass Spectrometry, J. Am. Soc. Mass Spectrom. 26, 1713-1721) in aqueous acetonitrile in the presence of triethylamine, followed by acidification to remove the ethylene acetal protecting group.

Synthesis of CAX-CHO. 4-Hydroxybenzaldehyde (Sigma Aldrich) with 1.1 equivalent of ethylene glycol and 0.1 equivalent of tosic acid in toluene is refluxed with a Dean Stark Trap. Workup is done by minimum dilution with water and extraction with toluene, followed by evaporation. The 4-hydroxybenzaldehyde ethylene acetal is reacted with CAX-B in aqueous acetonitrile in the presence of triethylamine, followed by acidification to remove the ethylene acetal protecting group, to yield the product.

### EXAMPLES

Example 1. Labeling of targeted purine(s), bearing an exocyclic primary amine group, with CAX-CHO, and use of a solid phase leashed amine to remove residual CAX-CHO. The said damaged purine(s), dissolved in 5 uL of LCMS-grade acetonitrile, are combined with 2 μL of acetonitrile containing 5 ug of CAX-CHO, and 2 μL of LCMS-grade methanol containing 60 μg of NaCNBH₃ in an Eppendorf tube that is then capped. After the resulting sample is vortexed for 10 seconds, it is kept at room temperature overnight. Adding the bead-leashed primary amine reagent of FIGS. 5 and 30 ug of NaCNBH₃ and continuing the incubation removes residual CAX-CHO.

Example 2. Labeling N3-adenines with CAX-Br, and use of a bead-leashed guanine (one of the options in FIG. 5) to remove residual CAX-B. The evaporated N3-adenines in a vial are redissolved in 6 uL of 50% acetonitrile containing CAX-B (prepared as described: Wang et al., [2015] Cationic Xylene Tag for Increasing Sensitivity in Mass Spectrometry, J. Am Soc. Mass Spectrometry, 26, 173-1721) at 1 mg/mL and 10 μL/mL of triethylamine, and the vial is capped, placed in the dark, and kept for 16 hours at 38° C. Ten mg of silica-leashed guanine (prepared as described: Poma et al., [2014] Nucleoside-Tailored Molecularly Imprinted Polymeric Nanoparticles (MIP NPs), Macromolecules, 47, 6322-6330) are added, and the vial is placed on a rotator plate followed by keeping for 16 more hours at 38° C. Adding the bead-leashed guanine reagent of FIG. 5 and continuing the incubation removes residual CAX-B. The CAX-labeled N3-adenines are recovered in the filtrate after ultrafiltration.

Example 3. Preparation of the bidentate diacyl phenyl type affinity material shown in FIGS. 3 and 4, and analogs, by multistep or combinatorial chemistry. 2-Nitrophthaloyl chloride (1 g) is dissolved in acetonitrile (30 mL) and 30 mL of acetonitrile containing one or more of reagents (or analogs of these reagents) such as ethanolamine, 3-amino-1-propanol, 4-amino-1-butanol, and 5-amino-1-butanol (100 mg each) along with 10 ml of triethylamine, is added slowly at ice bath temperature. A second round can use a second such reagent. The mixture is stirred in the dark at room temperature overnight. The mixture is dissolved in water or aqueous methanol and applied to the bead-immobilized guanine reagent of FIG. 5. Nonbinding compounds are washed out, and binding compounds are washed out subsequently and identified by NMR and mass spectrometry. The strongest binding compound is dissolved in 30 mL of 50% methanol, and the nitro group is reduced to an amino group with NaBH₄ as described (Pina et al., Reduction of nitrobenzene derivatives using sodium borohydride and transition metal sulfides [2014] Tetrahedron Letters, 55, 5408-5470). This product is then immobilized by reductive amination as shown in FIG. 5, for use as an affinity reagent to bind the II side (see FIG. 2) of purines unmodified on that side.

Example 4. Preparation of a bidentate diamino phenyl affinity material by multistep or combinatorial chemistry for binding the II side (see FIG. 2) of guanines unmodified on that side. 2-Nitro-para-phenylenediamine in acetonitrile or acetonitrile/methanol is reacted with a mixture of R—N-hydroxysuccinimide esters, where R comprises an alkyl, alkenyl, or aryl moiety; R contains 10 or fewer carbon atoms, and R can contain one or more functional groups such as OH, methoxy, ether, ketone, amide, secondary amine, tertiary amine, or thioether. These NHS esters are prepared by reacting RCO₂H with N-hydroxysuccinimide in the presence of N-ethyl-N′-(dimethylaminopropyl)carbodiimide hydrochloride in water. The reaction of the R—N-hydroxysuccinimide esters with the 2-nitro-para-phenylenediamine is conducted in aqueous buffer at pH 8.3-8.5, or in an organic solvent such as DMF or DMSO in the presence of one equivalent of a tertiary amine base such as triethylamine. The mixture, after evaporation, is dissolved in water or aqueous methanol and applied to the immobilized guanine reagent of FIG. 5. Nonbinding compounds are washed out, and binding compounds are washed out subsequently and identified by NMR and mass spectrometry. The strongest binding compound is dissolved in 30 mL of 50% methanol, and the nitro group is reduced to an amino group with NaBH₄ as described (Pina et al., Reduction of nitrobenzene derivatives using sodium borohydride and transition metal sulfides [2014] Tetrahedron Letters, 55, 5408-5470). This product is then bead-immobilized by reductive amination as shown in FIG. 5, for use as an affinity reagent to bind the II side (see FIG. 2) of purines unmodified on that side.

Example 5. Affinity chromatography. Affinity chromatography is performed in methanol: water, 1:10, v/v, or a similar solvent, by preparing a cartridge solid phase extraction column with the affinity material of interest, applying the sample of interest in this solvent, washing off unbound material with this or another solvent, and eluting bound material with this or another solvent.

Example 6. Preparation of an affinity reagent recognizing side III (see FIG. 2) of guanine. A ligand such as 4-aminophthalimide, 1-carboxymethyluracil, 6-carboxymethyluracil, 1-carboxymethylthymine, or N7-carboxymethylxanthine is used, by activation to an NHS ester that is then coupled to a solid support bearing primary amine groups. The thymine affinity reagent of Van Breemen et al., can be used (Van Breemen, R. B., Tan, Y., Lai, J., Huang, C.-R., Zhao, X. [1998] Immobilized thymine chromatography-mass-spectrometry of oligonucleotides. J. Chromatogr. A 806, 67-76).

Example 7. Preparation and use of an antibody or nanobody affinity reagent that recognizes PhIP, a genotoxic chemical from grilled red meat. N-(2′-deoxyguanosin-8-yl)-PhIP is prepared as described (Guo, J., Koopmeiners, S., Walmsley, S. J., Villalta, P. W., Yao, L., Murugan, P., Tejpaul, R., Weight, C. J., Turesky, R. J. The Cooked Meat Carcinogen 2-Amino-1-methyl-6-phenylimidazole[4,5-b]pyridine Hair Dosimeter, DNA Adductomics Discover, and Associations with Prostate Cancer Pathology Biomarkers, Chemical Research in Toxicology, https://doi.org/10.1021/acs.chemrestox.2c00012). This compound is then conjugated to 4-carboxybenzaldehyde in the presence of sodium cyanoborohydride; the resulting product is converted into an NHS ester as described in Example 4, followed by reaction with albumin to give the antigen, PhIP-albumin, having PhIP as a hapten, for production of an anti-PhIP antibody or nanobody This antibody is reacted with extended biotin-NHS, allowing the antibody to be subsequently immobilized on a chromatographic streptavidin column. The resulting column then is used as an affinity reagent to isolate PhIP-guanines from urine, which in turn are detected by CAX-CHO labeling followed by CAX-CHO removal as in Method A prior to detection by mass spectrometry.

This invention can also be practiced with a fluorescent dye bearing an aldehyde group, such as fluorescein aldehyde (Ettenauer, J., Semak, V., Brandl, M. (2018) Synthesis of Fluorescein Aldehydes for the Sensitive Detection of L-Cysteine, Proceedings 2, 895), fluorescein PEG aldehyde, or 3-(2-furoyl)quinoline-2-carboxaldehyde, where fluorescent detection would be done as by capillary electrophoresis with fluorescence detection, high performance liquid chromatography with fluorescent detection, or array fluorescence detection.

What is claimed is:

1. A method detecting a nucleobase, comprising the steps of:
a) subject a biosample or biosample extract containing noncanonical DNA to strong anion exchange extraction,
b) incubate the biosample or biosample extraction or from the anion exchanger to elute a nucleobase from the DNA,
c) covalently label the nucleobase with a positively charged reagent possessing a reactive aldehyde or alkylating group, yielding a tagged nucleobase, and
d) detect the tagged nucleobase by mass spectrometry.

2. The method of claim 1, wherein the nucleobase comprises a primary amino group which is labeled with an aldehyde-bearing mass tag having a positive charge, and the labeling is performed in the presence of sodium cyanoborohydride or pyridine borane.

3. The method of claim 1 or 2, wherein the aldehyde-bearing mass tag is affixed to the nucleobase using a reagent that contains a group selected from the group consisting of quaternary amine, tertiary amine, phosphonium, and pyridinium.

4. The method of any of claims 1-3, further comprising performing an affinity step immediately after the incubation step b), where a targeted or nontargeted nucleomer undergoes a noncovalent, multivalent binding interaction with a complementary group on a dissolved or undissolved material, and a targeted nucleomer is recovered.

5. The method of claim 4, wherein the affinity step is a partner affinity step.

6. The method of any one of claims 1-5, wherein the incubation step comprises mild neutral thermal hydrolysis or mild acid depurination.

7. The method of any one of claims 1-6, wherein the positively charged reagent is CAX-CHO or CAX-CHO-2.

8. A method of detecting a nucleomer having a primary amine group, comprising the steps of:
a) exposing a biosample or biosample extract to an affinity reagent, wherein the biosample or biosample extract comprises a targeted nucleomer that is primary-amine bearing and noncanonical;
b) recovering the targeted nucleomer and covalently reacting a primary amine group on the nucleomer with a positively charged reagent bearing an aldehyde group in the presence of a hydride reagent;
c) obtaining a conjugate having an amino-alkyl linkage, which is detected by mass spectrometry.

9. The method of claim 8, wherein the positively charge reagent comprises a group selected from the group consisting of quaternary amine, tertiary amine, pyridinium and phosphonium.

10. The method of claim 8 or 9, wherein the nucleomer is a nucleobase.

11. The method of claim 8 or 9, wherein the nucleomer is a nucleoside.

12. The method of any one of claims 8-11, wherein the biosample is urine.

13. The method of any one of claims 8-12, wherein the hydride reagent is sodium cyanoborohydride or pyridine borane.

14. The method of any one of claims 8-13, wherein the affinity reagent is selected from the group consisting of a canonical nucleomer, noncanonical nucleomer, diacylphenyl moiety, diaminophenyl moiety, β-cyclodextrin moiety, molecular imprinted polymer, nucleic acid aptamer, peptide aptamer, nanobody, antibody, and a riboswitch.

15. The method of claim 14, wherein the affinity reagent is a mixed-mode or semipolar chromatographic material.

16. The method of any one of claims 8-15, wherein the affinity step is conducted below 10° C.

17. The method of any one of claims 8-16, wherein a nontargeted nucleobase is extracted in the affinity step.

18. A method of detecting a nucleomer having a secondary amine, comprising the steps of:
a) exposing a biosample or biosample extract to an affinity reagent wherein the biosample or biosample extract comprises a targeted nucleomer that is secondary-amine bearing and noncanonical;
b) recovering the targeted nucleomer and covalently reacting a secondary amine group on the nucleomer with a positively charged reagent bearing an alkylating group;
c) obtaining a conjugate having an amino-alkyl linkage, which is detected by mass spectrometry.

19. The method of claim 18, wherein the nucleomer is a nucleobase.

20. The method of claim 18, wherein the nucleomer is a nucleoside.

21. The method of any one of claims 18-20, wherein the biosample is urine.

22. The method of any one of claims 18-21, wherein the positively charged reagent contains a quaternary amine group.

23. The method of any one of claims 18-22, wherein the alkylating group is a benzyl bromide, a xylyl bromide, a xylyl acetate, a xylyl tosylate, or an alkyl-chloride, -bromide, -iodide, or -tosylate.

24. The method of any one of claims 18-23, wherein the affinity reagent is selected from the group consisting of a canonical nucleomer, noncanonical nucleomer, diacylphenyl moiety, diaminophenyl moiety, β-cyclodextrin, molecular-imprinted polymer, nucleic acid aptamer, peptide aptamer, nanobody, antibody, aptabody, and a riboswitch.

25. The method of any one of claims 18-24, wherein the alkylation reaction is conducted in the presence of a tertiary amine.

26. The method of any one of claims 18-25, wherein the affinity reagent is a mixed-mode or semipolar chromatographic material.

27. The method of any one of claims 18-26, wherein the affinity step is conducted below 10° C.

28. The method of any one of claims 18-27, wherein a nontargeted nucleomer is extracted in the affinity step.

29. A method of detecting a compound bearing a primary amino group, comprising the steps of:
a) reacting the primary amino group with a positively charged reagent bearing a reactive aldehyde group in the presence of a hydride reagent, giving a conjugate having an amino-alkyl linkage; and
b) detecting the conjugate by mass spectrometry.

30. The method of claim 29, wherein the positively charged reagent is CAX-CHO or CAX-CHO-2.

31. The method of claim 29 or 30, wherein the compound is a nucleomer.
